(19) United States
(12) Patent Application Publication
(10) Pub. No.: US 2014/0046415 A1
(43) Pub. Date: Feb. 13, 2014

(54) REINFORCED COIL CREATED FROM POLYMER COATED WIRE FOR IMPROVED TORQUE TRANSFER

(71) Applicant: CARDIAC PACEMAKERS, INC., St. Paul, MN (US)

(72) Inventor: Devon N. Arnholt, Shoreview, MN (US)

(73) Assignee: CARDIAC PACEMAKERS, INC., St. Paul, MN (US)

(21) Appl. No.: 13/834,548

(22) Filed: Mar. 15, 2013

Related U.S. Application Data
(60) Provisional application No. 61/681,494, filed on Aug. 9, 2012.

(51) Int. Cl. A61N 1/05

(52) U.S. Cl. CPC A61N 1/05
USPC 607/116; 29/605

(57) ABSTRACT

An implantable medical device lead includes a lead body including a lumen extending from a proximal end of the lead body to a distal end of the lead body, and a helically coiled conductor including one or more filars extending through the lumen and including a plurality of turns. The implantable medical device lead further includes an insulative coating on at least one of the one or more filars, the insulative coating circumferentially covering the outer surface of the at least one of the one or more filars, and at least one cohesive structure formed between adjacent turns of the helically coiled conductor. The at least one cohesive structure includes portions of the insulative coating on the at least one of the one or more filars and is configured to interconnect adjacent turns of the helically coiled conductor.

### CROSS-REFERENCE TO RELATED APPLICATION

This application claims priority under 35 U.S.C. §119 to U.S. Provisional Application No. 61/681,494, filed on Aug. 9, 2012, entitled “REINFORCED COIL CREATED FROM POLYMER COATED WIRE FOR IMPROVED TORQUE TRANSFER,” which is incorporated herein by reference in its entirety for all purposes.

### TECHNICAL FIELD

The present disclosure relates to implantable medical devices. More particularly, the present disclosure relates to a medical device lead including a helically coiled conductor and one or more cohesive structures formed between adjacent turns of the helically coiled conductor.

### BACKGROUND

Implantable medical devices for treating a variety of medical conditions with electrical stimuli can include a medical electrical lead for delivering an electrical stimulus to a targeted site within a patient's body such as, for example, a patient's heart or nervous system. Some leads have an elongated, flexible insulating body, one or more inner conductors extending through lumens formed in the body and one or more exposed electrodes connected to the distal ends of the conductors.

Leads may be introduced into the patient's vasculature at a venous access site and transvenously guided through veins to the sites where the lead electrodes will be implanted or otherwise contact tissue at the targeted therapy site. A pulse generator attached to the proximal ends of the conductors delivers an electrical stimulus therapy to the targeted site via the one or more conductors.

Leads may include a fixation device configured to fixate the distal end of the lead at the therapy site. One or more of the conductors may be configured to transmit torque from the proximal end of the lead to the fixation device for driving the fixation device.

### SUMMARY

Discussed herein are implantable medical device leads including a helically coiled conductor and at least one cohesive structure formed between adjacent turns of the helically coiled conductor. Medical device lead conductors and also methods for producing a helically coiled conductor for a medical device are presented.

In Example 1, an implantable medical device lead includes a lead body including a lumen extending from a proximal end of the lead body to a distal end of the lead body, and a helically coiled conductor having a plurality of turns and including one or more filars extending through the lumen. The implantable medical device lead further includes an insulative coating on at least one of the one or more filars. The insulative coating circumferentially covers an outer surface of the at least one of the one or more filars. The implantable medical device lead also includes at least one cohesive structure formed between adjacent turns of the helically coiled conductor including portions of the insulative coating on the at least one of the one or more filars. The at least one cohesive structure is configured to interconnect adjacent turns of the helically coiled conductor.

In Example 2, the implantable medical device lead according to Example 1, wherein the at least one cohesive structure consists of the insulative coating.

In Example 3, the implantable medical device lead according to either Example 1 or Example 2, wherein the portions of the insulative coating of the at least one cohesive structure are fused together or welded together

In Example 4, the implantable medical device lead according to any of Examples 1-3, wherein the at least one cohesive structure is configured to continuously fill a region between adjacent turns of the helically coiled conductor, wherein the region is defined by the outer surfaces of the one or more filars bordering on the region.

In Example 5, the implantable medical device lead according to any of Examples 1-4, wherein the portions of the insulative coating are interconnected by polymer chains crossing interfaces between the portions of the insulative coating of the at least one cohesive structure.

In Example 6, the implantable medical device lead according to any of Examples 1-5, wherein each of the filars of the helically coiled conductor comprises an insulative coating circumferentially covering the outer surface of each of the filars.

In Example 7, the implantable medical device lead according to Example 6, wherein the minimum width of the at least one cohesive structure (measured in a direction parallel to the center axis of the helically coiled conductor) is less than the sum of the thicknesses of a first section of the insulative coating covering the outer surface of a first filar which borders on the cohesive structure and a second section of the insulative coating covering the outer surface of a second filar which borders on the cohesive structure, said first and second sections of the insulative coating facing a center axis of the helically coiled conductor.

In Example 8, the implantable medical device lead according to Example 6, wherein the minimum width of the at least one cohesive structure (measured in a direction parallel to the center axis of the helically coiled conductor) is greater than the sum of the thicknesses of a first section of the insulative coating covering the outer surface of a first filar which borders on the cohesive structure and a second section of the insulative coating covering the outer surface of a second filar which borders on the cohesive structure, said first and second sections of the insulative coating facing a center axis of the helically coiled conductor.

In Example 9, the implantable medical device lead according to any of Examples 1-5, wherein only one of any two adjacent filars comprises an insulative coating circumferentially covering the outer surface of the filar.

In Example 10, the implantable medical device lead according to Example 9, wherein the minimum width of the at least one cohesive structure, measured in a direction parallel to the center axis of the helically coiled conductor, is less than the thickness of a section of the insulating coating facing a center axis of the helically coiled conductor and covering the outer surface of a filar that borders on the cohesive structure.

In Example 11, the implantable medical device lead according to Example 9, wherein the minimum width of the at least one cohesive structure, measured in a direction parallel to the center axis of the helically coiled conductor, is greater than the thickness of a section of the insulative coating facing a center axis of the helically coiled conductor and covering the outer surface of a filar that borders on the cohesive structure.

In Example 12, the implantable medical device lead according to any of Examples 1-11, wherein the at least one cohesive structure is co-radially and co-axially coiled with the one or more filars.

In Example 13, the implantable medical device lead according to any of Examples 1-12, wherein a minimum width of the at least one cohesive structure, measured in a direction parallel to the center axis of the helically coiled conductor, is in the range of about 0.0005 inch to about 0.008 inch.

In Example 14, the implantable medical device lead according to any of Examples 1-13, wherein the insulative coating comprises a polymer, a thermoplastic or a thermoplastic elastomer, expanded polytetrafluoroethylene (ePTFE), layered ePTFE, polytetrafluoroethylene (PTFE), polyethylene terephthalate (PETE), ethylene/tetrafluoroethylene copolymer (ETFE), fluorinated ethylene propylene (FEP), polyether ether ketone (PEEK), polyamides, polyimides, para-aramid synthetic fibers, and polyurethane.

In Example 15, the implantable medical device lead according to any of Examples 1-14, wherein the implantable medical device lead further includes a polymer sheath formed about the helically coiled conductor.

In Example 16, the implantable medical device lead according to Example 15, wherein the polymer sheath comprises a material different than the insulative coating.

In Example 17, the implantable medical device lead according to Example 16, wherein the material of the polymer sheath has a melting temperature or a glass transition temperature lower than a melting temperature or a glass transition temperature of the insulative coating.

In Example 18, the implantable medical device lead according to any of Examples 1-17, wherein the implantable medical device lead further includes a fixation device connected to a distal end of the helically coiled conductor.

In Example 19, a medical device lead conductor includes at least one helically coiled conducting filar including a plurality of filar turns. At least one of any two adjacent filar turns have a coating circumferentially covering the outer surface of the at least one filar turn with at least one coating material. The conductor further includes at least one cohesive structure bordering the outer surfaces of any two adjacent filar turns and consisting of merged portions of the at least one coating material. The at least one cohesive structure is configured to interconnect pairs of adjacent filar turns and to increase the torsional stiffness of the at least one helically coiled conducting filar.

In Example 20, a method for producing a helically coiled conductor for a medical device includes forming an insulative coating over at least one of one or more filars, coiling the one or more filars into a plurality of co-radial turns, and softening the insulative coating such that adjacent turns of the one or more filars interconnect with one another.

In Example 21, the method according to Example 20, wherein after coiling and prior to softening the method further includes forming a sleeve around an outer diameter of the one or more coiled filars.

In Example 22, the method according to Example 21, wherein the sleeve is configured to exert radial compression forces on the one or more filars during the softening step and/or to enhance a flow of portions of the insulative coating into regions located between adjacent turns during the softening step.

In Example 23, the method according to any of Examples 20-22, wherein the softening step softens the insulative coating such that portions of the insulative coating flow into regions located between adjacent turns and accumulate in these regions thereby forming at least one cohesive structure interconnecting the adjacent turns.

In Example 24, the method according to any of Examples 20-23, wherein the method further includes the step of forming a polymer sheath over the at least one coiled filar.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic view of a cardiac rhythm management system including an implantable medical device coupled to a lead deployed in a patent's heart.

FIG. 2A is a schematic view of an implantable medical device lead including a helically coiled conductor.

FIG. 2B is a cross-sectional view of the implantable medical device lead shown in FIG. 2A.

FIG. 2C is a further cross-sectional view of the implantable medical device lead shown in FIG. 2A.

FIG. 3A is a perspective view of a helically coiled conductor of the implantable medical device lead shown in FIG. 2A.

FIG. 3B is a cross-sectional view of the helically coiled conductor shown in FIG. 3A.

FIG. 3C is a detailed cross-sectional view of the helically coiled conductor shown in FIG. 3A.

FIG. 4A is a perspective view of a helically coiled conductor of an implantable medical device lead.

FIG. 4B is a cross-sectional view of the helically coiled conductor shown in FIG. 4A.

FIG. 4C is a detailed cross-sectional view of the helically coiled conductor shown in FIG. 4A.

FIG. 5A is a diagram showing the torque transmitted by helically coiled conductors of implantable medical device leads as a function of revolutions of the conductors.

FIG. 5B is a diagram showing the torque transmitted by a helically coiled conductors of implantable medical device leads as a function of revolutions of the conductors.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

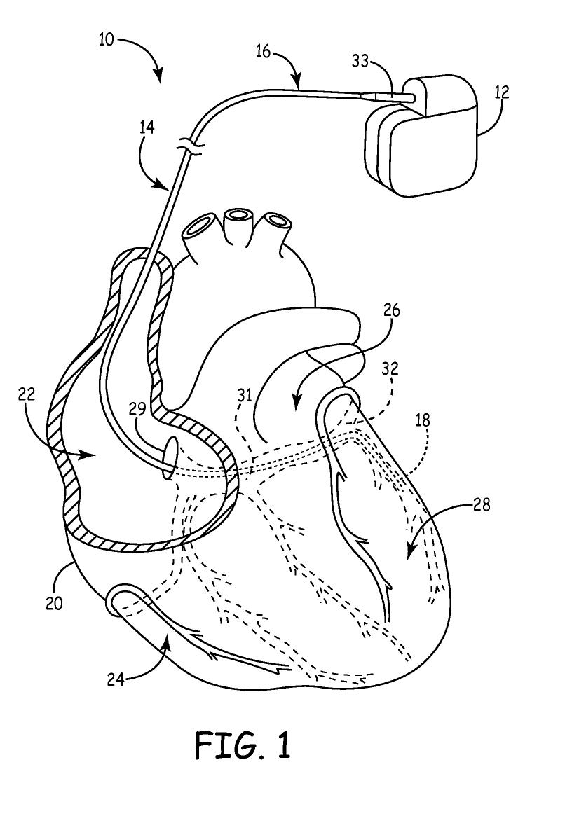

FIG. 1 is a schematic view of a cardiac rhythm management system 10 including an implantable medical device (IMD) 12 with a lead 14 having a proximal end 16 and a distal end 18. In one embodiment, the IMD 12 includes a pulse generator (not shown) such as a pacemaker or a defibrillator. In one embodiment, the IMD includes a processing unit configured to process electrical signals sensed by the lead 14. The IMD 12 can be implanted subcutaneously within the body, typically at a location such as in the patient's chest or abdomen, although other implantation locations are possible. The proximal end 16 of the lead 14 can be coupled to or formed integrally with the IMD 12. The distal end 18 of the lead 14, in turn, can be implanted at a desired location in or near the heart 20.

As shown in FIG. 1, a distal portion of the lead 14 is disposed in a patient's heart 20, which includes a right atrium 22, a right ventricle 24, a left atrium 26, and a left ventricle 28. In the embodiment illustrated in FIG. 1, the distal end 18 of the lead 14 is transvenously guided through the right atrium 22, through the coronary sinus ostium 29, and into a branch of the coronary sinus 31 or the great cardiac vein 32. The illustrated position of the lead 14 can be used for sensing or for delivering pacing and/or defibrillation energy to the left side of the heart 20, or to treat arrhythmias or other cardiac disorders requiring therapy delivered to the left side of the heart 20. Additionally, it will be appreciated that the lead 14 can also be used for sensing or to provide treatment in other regions of the heart 20 (e.g., the right ventricle 24), or to other areas of the body (e.g., nerves).

Although the illustrative embodiment depicts only a single implanted lead 14, it should be understood that multiple leads can be utilized so as to sense or electrically stimulate other areas of the heart 20. In some embodiments, for example, the distal end of a second lead (not shown) may be implanted in the right atrium 22, and/or the distal end of a third lead (not shown) may be implanted in the right ventricle 24. Other types of leads such as epicardial leads may also be utilized in addition to, or in lieu of, the lead 14 depicted in FIG. 1.

During operation, the lead 14 can be configured to convey electrical signals between the IMD 12 and the heart 20. For example, in those embodiments where the IMD 12 is a pacemaker, the lead 14 can be utilized to deliver electrical stimuli for pacing the heart 20. In those embodiments where the IMD 12 is an implantable cardiac defibrillator, the lead 14 can be utilized to deliver electric shocks to the heart 20 in response to an event such as a heart attack or arrhythmia. In some embodiments, the IMD 12 includes both pacing and defibrillation capabilities. The lead can also be configured to sense electrical signals indicative of a physiological condition of the heart 20 and to convey sensed signals to the IMD 12.

The electrical signals are carried between the IMD 12 and electrodes at the distal end 18 by one or more conductors extending through the lead 14. The one or more conductors are electrically coupled to a connector 33 suitable for interfacing with the IMD 12 at the proximal end 16 of the lead 14, and to one or more electrodes at the distal end 18.

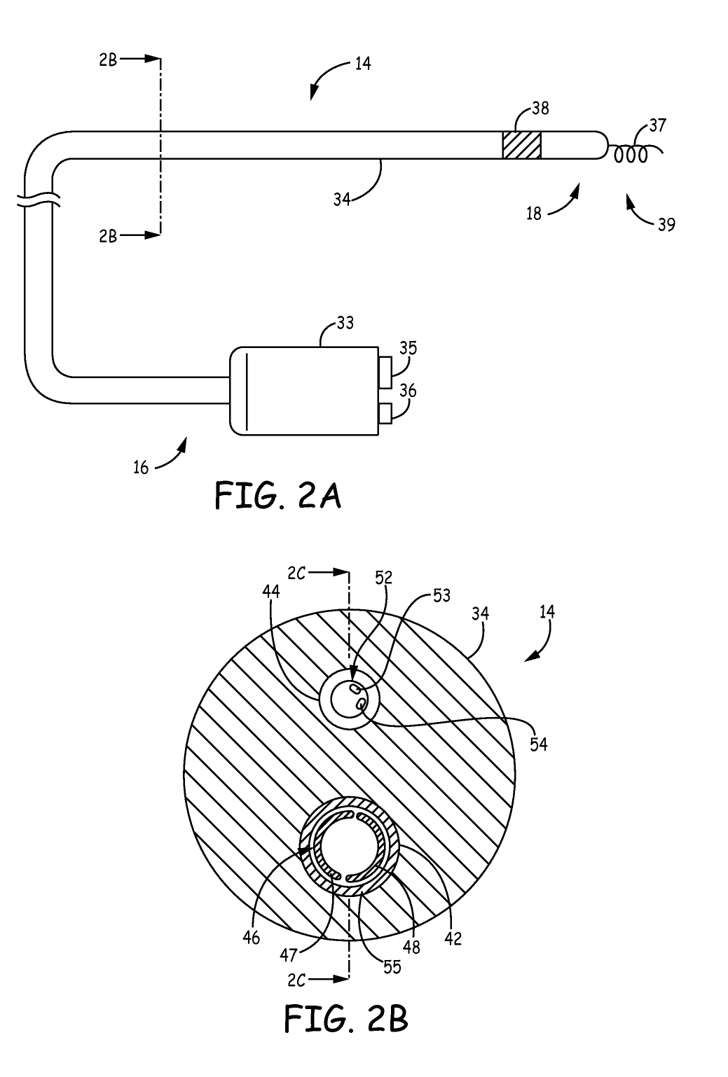

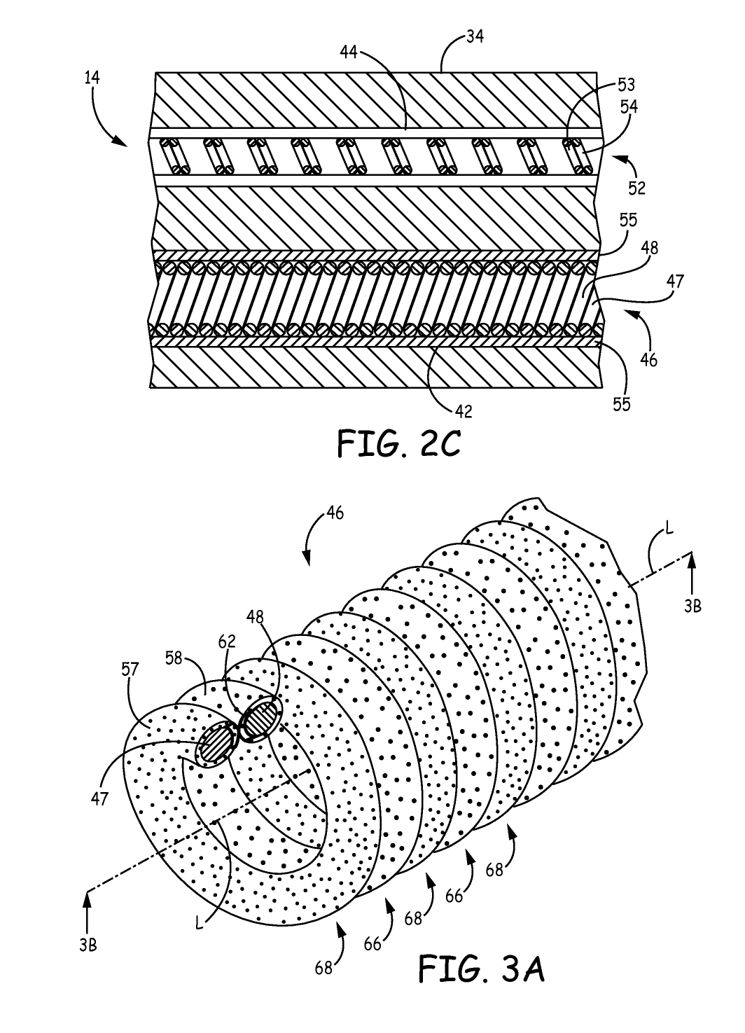

FIG. 2A is a schematic view of the lead 14 shown in FIG. 1. The lead 14 includes a lead body 34. At the proximal end 16, the lead body 34 carries the connector 33 with electrical contacts 35 and 36 adapted for electrically and mechanically coupling the lead 14 to the IMD 12 shown in FIG. 1.

The lead further includes a tip electrode 37 and a ring electrode 38 located at the distal end 18 of the lead body 34 of the lead 12. In one embodiment, the electrodes 37, 38 are configured to apply therapy pulses to tissue in contact with these electrodes 37, 38 and/or to sense electrical signals indicative of a physiological condition. The implantable medical device lead 14 further includes a fixation device 39 at the distal end 18 of the lead body 34 configured to fixate the distal end 18 of the lead body 34 to heart tissue. The tip electrode 37 is helically shaped and adapted as connector element of the fixation device 39. In some embodiments, the fixation device 39 may further include a mechanism (not shown) located within the lead body 34 for extending the connector element, i.e. tip electrode 37, from the lead body 34, retracting the connector element into the lead body 34 and for rotating the connector element, i.e. tip electrode 37, relative to the lead body 34. The mechanism may be configured to be drivable by means of torque transmitted by one of the conductors of the lead 14.

FIG. 2B shows a cross-section of FIG. 2A and FIG. 2C shows a cross-section of FIG. 2B. The lead body 34 includes a first lumen 42 and a second lumen 44. In some embodiments, both lumens 42, 44 extend from the proximal end 16 to the distal end 18 of the lead 14. A helically coiled conductor 46 includes a first conductive filar 47 and a second conductive filar 48 forming a plurality of turns of the conductor 46 and extending through the first lumen 42 of the lead body 34. The filars 47, 48 electrically connect the contact 35 of the connector 33 with the tip electrode 37. In some embodiments, a polymer sheath 55 is formed about the helically coiled conductor 46. A further helically coiled conductor 52 also includes a first conductive filar 53 and a second conductive filar 54 forming a plurality of turns and extending through the second lumen 44. The filars 53, 54 electrically connect the contact 36 of the connector 33 of the lead 14 with the ring electrode 38 of the lead 14. While conductors 46 and 52 each include two filars in the illustrated embodiment, the conductors 46 and/or 52 may alternatively include one filar or more than two filars.

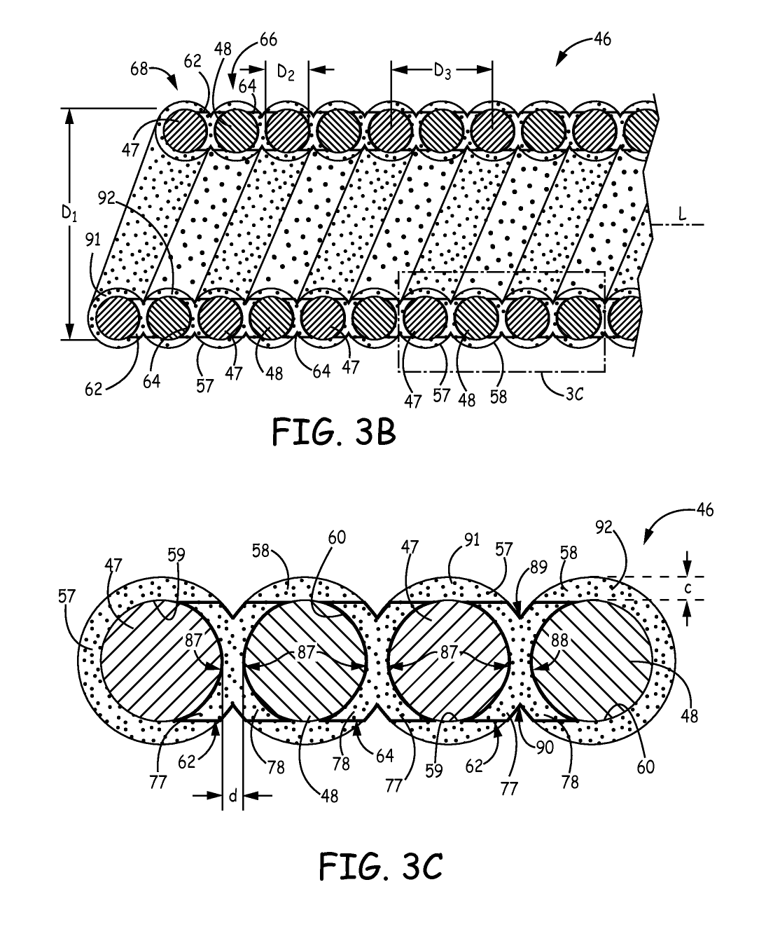

FIG. 3A shows a perspective view of the helically coiled conductor 46 of the implantable medical device lead 14 shown in FIGS. 2A to 2C. FIG. 3B shows a cross-section of FIG. 3A. FIG. 3C shows a detailed view of the section of FIG. 3B indicated by dotted lines. The implantable medical device 14 lead includes a first insulative coating 57 on the first filar 47 of the conductor 46 and a second insulating coating 58 on the second filar 48. In some embodiments, the insulative coatings 57, 58 are polymers, such as thermoplastics or thermoplastic elastomers. For instance, the insulative coating 57 may be ethylene/tetrafluoroethylene copolymer (ETFE) and the insulative coating 58 may be polytetrafluoroethylene (PTFE). Alternatively, other materials are also possible, including, but not limited to, a polymer, a thermoplastic or a thermoplastic elastomer, expanded polytetrafluoroethylene (ePTFE), layered ePTFE, polytetrafluoroethylene (PTFE), polyethylene terephthalate (PETE), ethylene/tetrafluoroethylene copolymer (ETFE), fluorinated ethylene propylene (FEP), polyether ether ketone (PEEK), polyamides, polyimides, para-aramid synthetic fibers, and polyurethane. In some embodiments, the materials of the insulative coatings 57, 58 on adjacent filars 47, 48 are identical to each other. In some embodiments, the materials of the insulative coatings 57, 58 on adjacent filars 47, 48 are different from each other.

As shown in FIG. 3C, the first insulative coating 57 circumferentially, i.e. continuously and all-around, covers the outer surface 59 of the first filar 47 and the second insulative coating 58 circumferentially covers the outer surface 60 of the second filar 48. The filars 47 and 48 are electrically isolated from each other by the insulative coatings 57 and 58. The implantable medical device lead 14 also includes cohesive structures 62, 64 formed between adjacent turns 66, 68 of the helically coiled conductor 46. The cohesive structures 62 and 64 are co-radially and co-axially coiled with the filars 47, 48. Each of the cohesive structures 62, 64 consists of the insulative coatings 57, 58 and is configured to interconnect adjacent turns 66, 68 of the helically coiled conductor 46. The cohesive structures 62 thereby increase the torsional stiffness of the helically coiled conductor 46. In the illustrated embodiment, the polymer sheath 55 is not part of the cohesive structures 62 and 64 such that the cohesive structures 62 and 64 are free of any portions of the polymer sheath 55.

Portion 77 of the insulative coating 57 and portion 78 of the insulative coating 58 are fused together such that the cohesive structures 62 and 64 are formed and polymer chains (not shown) of the insulative coatings 57, 58 cross interfaces (not shown) between the portions 77, 78. Both end pieces of any of these polymer chains can be connected with other polymer chains (not shown) completely residing within one of these portions 77, 78. In this way, the polymer chains of the fused portions 77, 78 provide cohesive forces to interconnect the portions 77, 78 and in this way hold together the cohesive structure 62 as well as the cohesive structure 64. In some embodiments, any two of the adjacent turns 66, 68 of the helically coiled conductor 46 are interconnected by the one more cohesive structure 62, 64.

The cohesive structures 62, 64 are configured to transfer tangential forces between the adjacent turns 66, 68 in response to torque applied to the conductor 46. These tangential forces can suppress tangential sliding motions between the adjacent turns 66, 68 and thereby suppress both unwinding as well as further winding of the helically coiled conductor 46 depending on the direction of torque. To this end, the cohesive structures 62, 64 exhibit a sufficiently high shear viscosity provided by interconnected and entangled polymer chains within the cohesive structures 62, 64. At the same time, the cohesive structures 62, 64 are elastic and resilient such that only relatively small axial forces are transferred between adjacent turns 66, 68 of the conductor 46, said axial forces being parallel to the center axis L (FIG. 3A) of the helically coiled conductor 46. In this way, the bending properties of the conductor 46 are largely retained whereas the torsional stiffness of the conductor 46 is increased significantly.

In some embodiments, the torsional stiffness of the conductor 46 allows torque to be transmitted from the proximal end 16 to the distal end 18 of the lead 14 sufficient for screwing the fixation device 39 into tissue, such as heart tissue.

As shown in FIG. 3C, the cohesive structures 62, 64 can be configured to continuously fill regions between adjacent turns 66, 68 of the helically coiled conductor 46. Said regions are defined by the outer surfaces 59, 60 of the filars 47, 48 bordering on the regions. In this embodiment, there are no gaps within said regions or any or any further layers in addition to the insulating coatings 57, 58 forming the cohesive structures 62, 64. FIG. 3C shows cross-sectional areas of the cohesive structures 62, 64 defined by a longitudinal cut through the helically coiled conductor, the cutting plane of the longitudinal cut containing the center axis L of the helically coiled conductor 46, see FIG. 3A. For both of the cohesive structures 62, 64, the cross-sectional area has two first inward-curving, i.e. concave, surfaces 87, 88 contacting, i.e. bordering on, the outer surfaces 59, 60 of the filars 47, 48. The cross-sectional areas also have two second inward-curving surfaces 89, 90 not contacting the filars 47, 48 and located, with respect to said center axis L, substantially in the middle between said first inward-curving surfaces 87, 88. Due to the second inward-curving surfaces 89, 90 the cohesive structures 62, 64 each have a waist improving the bendability of the conductor 46.

In some embodiments, the minimum width d (measured in a direction parallel to the center axis L of the helically coiled conductor) of the cohesive structures 62, 64 is smaller than the sum of the thicknesses c of a first section 91 of the insulative coating 57 covering the first filar 47 and a second section 92 of the insulative coating 58 covering the second filar 48, said first and second coating sections 91, 92 facing the center axis L of the helically coiled conductor 46. In some embodiments, said minimum width d of the cohesive structures 62, 64 is greater than the sum of said thicknesses c. The minimum width d of the cohesive structures 62, 64 may be in the range of about 0.0005 inch to about 0.008 inch (0.0127-0.2032 mm). The thicknesses c of said sections 91, 92 of the insulative coatings 57, 58 may be equal or different from each other and each can be in the range of about 0 inch to about 0.004 inch (0-0.1016 mm). Small values of the minimum width d may result in small coil pitches and in better MRI compatibility. Larger values of the minimum width d of the cohesive structures 64, 62 may result in a higher torsional stiffness of the helically coiled conductor 46.

The outer diameter D₁ of the coiled filars 47, 48, the filar diameter D₂ of the coiled filars 47, 48 (defined without the insulative coatings 57, 58), and the coil pitch D₃ are selected to minimize effects of magnetic resonance imaging (MRI) scans on the functionality and operation of the lead 14. For example, the outer diameter D₁ of the helically coiled conductor may be in the range of about 0.002 inch to 0.05 inch (0.051-1.27 mm), the filar diameter D₂ of the filars 47, 48 may be in the range of about 0.0005 inch to about 0.011 inch (0.013-0.28 mm), and the coil pitch D₃ may be in the range of about one to two times the filar diameter D₂. In one exemplary implementation, the outer diameter D₁ is about 0.03 inch (0.76 mm), the filar diameter D₂ is about 0.003 inch (0.076 mm), and the coil pitch D₃ is about 0.005-0.006 inch (0.127-0.152 mm). The axial length of the helically coiled conductor is in the range of about 450 mm to 640 mm. In one exemplary implementation, the axial length is about 500 mm. In some embodiments, D₁, D₂, and D₃ are chosen such that a total inductance of the coiled conductor 46 is in the rage of about 1.0 μH to about 5.0 μH, preferably greater than 1.5 μH. In one exemplary implementation, the total inductance of the coiled conductor 46 is about 3.0 μH.

In some embodiments, the polymer sheath 55 (shown in FIGS. 2B and 2C) can have a thickness in the range of about 0.0001 inch to 0.003 inch (0.00254-0.762 mm), for instance 0.001 inch (0.0254 mm). The polymer sheath 55 can be configured to further increase the torsional stiffness of the helically coiled conductor 46 by exerting compressive radial forces on the turns 66, 68 of the conductor 46 when torque is applied to the conductor 46. These radial compressive forces suppress expansion of the helically coiled conductor in the radial direction in response to the applied torque. The polymer sheath 55 may also be configured to exert uncompressing radial forces on the turns of the conductor when torque is applied to the conductor 46 in an opposite direction as compared to the case described above. This may be achieved by bonding the polymer sheath 55 to the insulative coatings 57 and 58 of the conductor 46. The radial uncompressing forces suppress collapsing of the helically coiled conductor in radial direction in response to the applied torque. The radial forces exerted by the polymer sheath 46 and the tangential forces transferred by the cohesive structures 62, 64 complement one another and yield high torsional stiffness of the conductor 46.

In one embodiment, the helically coiled conductor 46 shown in FIGS. 2B-3C is produced by forming the insulative coatings 57, 58 over the filars 47, 48, subsequently coiling the filars 47, 48 into a plurality of co-radial turns 66, 68, and heating the insulative coatings 57, 58 to a temperature that softens the insulative coatings 57, 58. In some embodiments, the heating is such that the portions 77, 78 of the insulative coatings 57, 58 on adjacent turns 66, 68 of the filars 47, 48 are fused together, thereby forming the cohesive structures 77, 78 and interconnecting the adjacent turns 66, 68. Additionally, the softening step softens the insulative coatings 57, 58 such that portions of the insulative coatings 57, 58 flow into the regions between the adjacent turns 66, 68, accumulating in these regions and merging in the portions 77, 78 which form the cohesive structures 62, 64. The temperature for softening the insulative coatings 57, 58 can be sufficiently low, e.g., at or only slightly above a glass transition temperature of the insulating coatings 57, 58, such that the insulative coatings 57, 58 remain undamaged during the softening step.

After coiling the filars 47, 48 and prior to softening, a sleeve (not shown) can be formed around the outer diameter D₁ of the coiled filars 47, 48. The sleeve can be configured to exert radial compression forces on the filars 47, 48 to mechanically stabilize the filars 47, 48 during the softening step. In some embodiments, the sleeve is configured to enhance and/or direct the flow of the portions of the softened insulative coating 57, 58 into the regions located between adjacent turns 66. 68. For those purposes, the sleeve does not melt or significantly soften during the softening step. After forming the cohesive structures 77, 78 the sleeve may be removed from the filars 62, 64.

After removing the sleeve, the polymer sheath 55 can be formed over the helically coiled conductor 46 by extruding the polymer sheath 55 over the conductor 46. Alternatively, the polymer sheath may also be formed by molding the sheath 55 around the coiled filars 47, 48, by adhering the sheath to the coiled filars 47, 48, or by heat shrinking the sheath over the coiled filars 47, 48. In some embodiments, the polymer sheath 55 can comprise a material different than the insulative coatings 57, 58, for instance polyamide. In some embodiments, the polymer sheath 55 can have a melting or glass transition temperature lower than the insulative coating 57, 58 so that the cohesive structures 77, 78 remain intact during the formation of the polymer sheath 55.

In an alternative embodiment, the sleeve is not removed but is retained around the filars 47, 48, e.g. as or in addition to the sheath 55, for increasing the torsional stiffness of the helically coiled conductor 46. In some embodiments, the insulative coatings 57, 58 bond to the sleeve or the sheath 55 during the softening step.

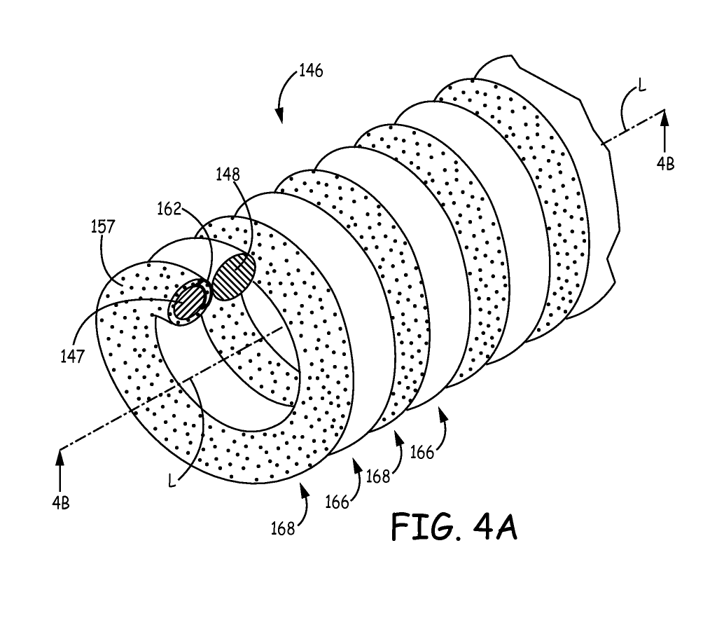

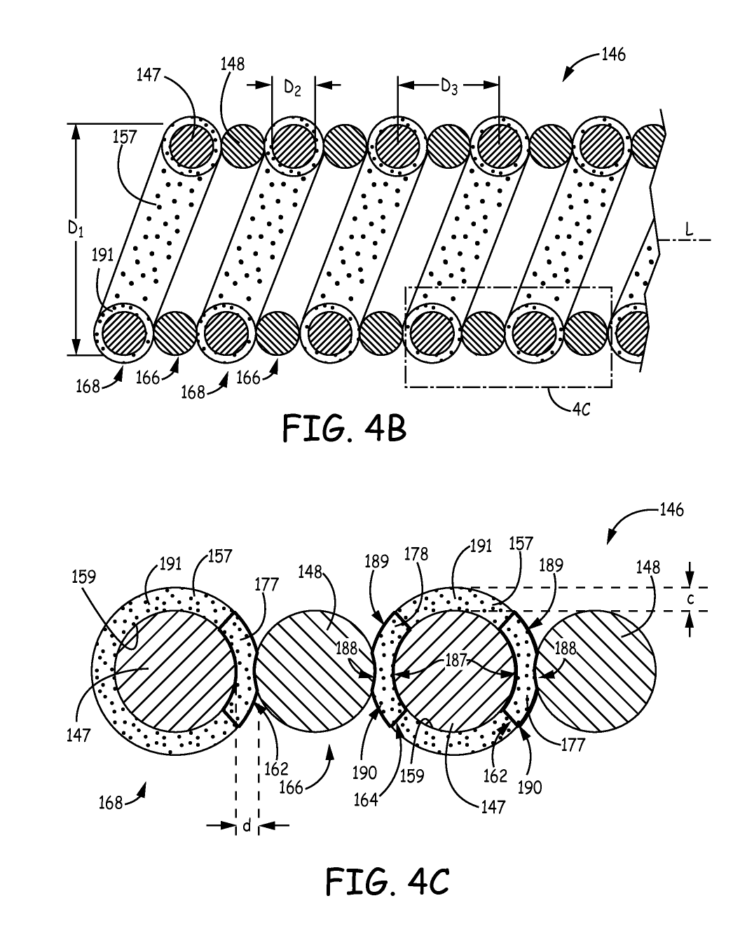

FIG. 4A shows a perspective view of a helically coiled conductor 146 according to a further embodiment of the present disclosure. FIG. 4B shows a cross-section of FIG. 4A. The helically coiled conductor 146 includes a first conductive filar 147 and a second conductive filar 148 forming a plurality of turns 166, 168 of the conductor 146. The helically coiled conductor 146 can be used, for example, with the implantable medical device lead 14 shown in FIGS. 2A to 2C replacing the helically coiled conductor 46. In some embodiments, the filars 147, 148 extend through the first lumen 42 of the lead body 34 of the lead 14 and electrically connect the contact 35 of the connector 33 with the tip electrode 37. In the illustrated embodiment, the first filar 147 includes an insulative polymer coating 157, such as a thermoplastic or a thermoplastic elastomer. In one exemplary implementation, the insulative coating 157 is ETFE. Alternatively, other materials are also possible, such as those materials listed herein with respect to insulative coatings 57, 58.

FIG. 4C shows a detailed view of FIG. 4B. The insulative coating 157 circumferentially covers the outer surface 159 of the first filar 147, while the second filar 148 does not include and insulative coating. In this embodiment, the implantable medical device lead 14 also includes cohesive structures 162, 164 formed between adjacent turns 166, 168 of the helically coiled conductor 146 and which are co-radially and co-axially coiled with the filars 147, 148. In the FIGS. 4A to 4C, the cohesive structures 162, 164 are indicated by thick marking lines surrounding the cohesive structures 162, 164. Each of the cohesive structures 162, 164 consists of a portion 177 or 178, respectively, of the insulative coating 157 on the filar 147 and is configured to interconnect adjacent turns 166, 168 of the helically coiled conductor 146.

The cohesive structures 162, 164 can be configured to increase the torsional stiffness of the helically coiled conductor 146 by transferring tangential forces between the adjacent turns 166, 168 in response to torque, (e.g., the torque vector oriented parallel or anti-parallel relative to the center axis L of the conductor 146) applied to the conductor 146. These tangential forces can suppress tangential sliding motion between the adjacent turns 166, 168 and thereby suppress both unwinding as well as further winding of the helically coiled conductor 146 under torsional stress. To this end, the cohesive structures 162, 164 can exhibit a sufficiently high shear viscosity provided by interconnected and entangled polymer chains within the cohesive structures 162. At the same time, the cohesive structures 162, 164 can be elastic and resilient such that only relatively small axial forces are transferred between adjacent turns 166, 168 of the conductor 146. The axial forces are parallel to the center axis L (FIG. 4A) of the helically coiled conductor 146. In this way, the bending properties of the conductor 146 can be largely retained.

In particular, the torsional stiffness of the conductor 146 improves transmission of torque from the proximal end 16 to the distal end 18 via the helically coiled conductor 146, sufficient for screwing the helically shaped connector element, i.e. the tip electrode 37 of lead 14, into tissue, such as heart tissue.

As shown in FIG. 4C, each of the cohesive structures 162, 164 can be configured to continuously fill regions between adjacent turns 166, 168 of the helically coiled conductor 146. Said regions are defined by the outer surfaces 159, 160 of the filars 147, 148, respectively. FIG. 4C shows cross-sectional areas of the cohesive structures 162, 164 which are defined by a longitudinal cut through the helically coiled conductor, the cutting plane of said longitudinal cut containing the center axis L of the helically coiled conductor (FIG. 4A). For both of the cohesive structures 162, 164, the cross-sectional area has two inward-curving, i.e. concave, surfaces 187, 188 contacting, i.e. bordering on, the outer surfaces 159, 160 of the filars 147, 148. The cross-sectional areas also have two outward-curving, i.e. convex, surfaces 189, 190 not contacting the filars 147, 148 and located, with respect to said center axis L, substantially in the middle between said inward-curving surfaces 187, 188. This configuration has the advantage of axial compactness and a small pitch for increased inductance. On the other hand, those embodiments wherein each of the filars comprises an insulative coating circumferentially covering the outer surfaces of the filars, as shown in FIGS. 3A-3C, may exhibit very high torsional stiffness.

The minimum width d (measured in a direction parallel to the center axis L of the helically coiled conductor) of the cohesive structures 162, 164 is smaller than the thickness c of a section 191 of the insulative coating 157 covering the first filar 147 and facing the center axis L of the helically coiled conductor 146. In some embodiments, said minimum width d of the cohesive structures 162, 164 is greater than the thickness c of said section 191 of the insulative coating 157. In some embodiments, the minimum width d of the cohesive structures 162, 164 can be in the range of about 0.0005 inch to about 0.008 inch (0.0127-0.2032 mm). In some embodiments, the thickness c of the section 191 of the insulative coating 157 can be in the range of about 0 inch to about 0.004 inch (0-0.1016 mm).

The outer diameter D₁ of the coiled filars 147, 148, the filar diameter D₂ of the coiled filars 147, 148 (defined without the insulative coating 157), and the coil pitch D₃ can be selected to minimize effects of magnetic resonance imaging (MRI) scans on the functionality and operation of the lead 14 including the helically coiled conductor 146. In some embodiments, the outer diameter D₁, filar diameter D₂, and coil pitch D₃ of the filars 147, 148 can be similar to the corresponding dimensions of filars 47, 48 as described herein.

In one embodiment, the helically coiled conductor 146 shown in FIGS. 4A-4C is produced by forming the insulative coating 157 over the first filar 147, subsequently coiling the filars 147, 148 into a plurality of co-radial turns 166, 168, and heating the insulative coating 157 to a temperature that softens the insulative coating 157 such that portions 177, 178 of the insulative coating 157 form the cohesive structures 162, 164. The cohesive structures 162, 164 are located between adjacent turns 166, 168 of the filars 147, 148 and interconnect the turns 166, 168 of the filars 147, 148. Additionally, the softening step softens the insulative coating 157 such that portions of the insulative coating 157 flow into the regions between the adjacent turns 166, 168, accumulate in these regions and merge in the portions 177, 178 which form the cohesive structures 162, 164. The temperature for softening the insulative coating 157 can be low enough to prevent damage to the insulating coating 157 during the softening step.

A sleeve and/or a polymer sheath 55 can be formed around the coiled filars 147, 148 of conductor 146 in a similar way as described in detail above for the conductor 46 shown in FIGS. 2B-3C.

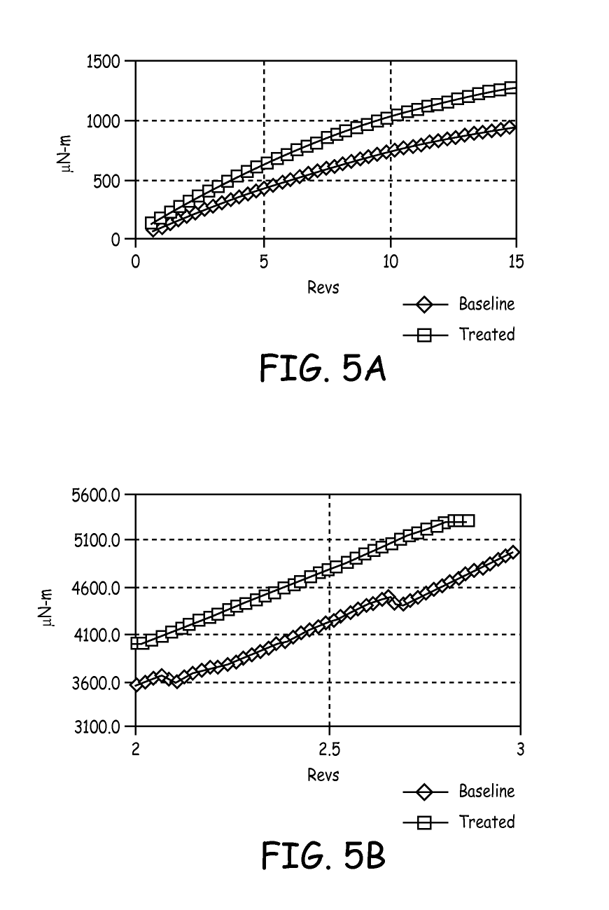

FIGS. 5A and 5B show diagrams of the torque (in units of μN·m) transmitted by a helically coiled conductors of implantable medical device leads as a function of the number of revolutions of one end of a conductor around the center axis of the conductor. Each diagram shows one curve labeled as “Treated” and corresponding to a first conductor according to one embodiment of the present disclosure, such as the conductors 46 or 146 discussed above, and one curve labeled “Baseline” and corresponding to a second conductor which is identical to the first conductor except that adjacent turns of the second conductor are not interconnected.

The diagrams of FIGS. 5A and 5B show that the torque transmitted by the first conductor according to one embodiment of the present disclosure is significantly larger than the torque transmitted by the second conductor. For example, for 10 revolutions, the torque transmitted by the first conductor is about 130 percent of the torque transmitted by the second conductor, and for 2.5 revolutions, the torque transmitted by the first conductor is about 115 percent of the torque transmitted by the second conductor. Furthermore, as can be seen from FIG. 5B, the torque transmitted by the first conductor is a smooth and monotonously increasing function of the number of revolutions, whereas the torque of the second conductor exhibits sudden drops resulting in local minima of the transmitted torque. This demonstrates that a conductor according to the present disclosure allows transmitting torque in a more controlled way than the second conductor.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. An implantable medical device lead comprising:
a lead body including a lumen extending from a proximal end of the lead body to a distal end of the lead body;
a helically coiled conductor including one or more filars extending through the lumen, the helically coiled conductor comprising a plurality of turns;
an insulative coating on at least one of the one or more filars, the insulative coating circumferentially covering an outer surface of the at least one of the one or more filars; and
at least one cohesive structure formed between adjacent turns of the helically coiled conductor, the at least one cohesive structure comprising portions of the insulative coating on the at least one of the one or more filars, wherein the cohesive structure is configured to interconnect adjacent turns of the helically coiled conductor.

2. The implantable medical device lead of claim 1, wherein the at least one cohesive structure consists of the insulative coating.

3. The implantable medical device lead of claim 1, wherein the portions of the insulative coating of the at least one cohesive structure are fused together or welded together.

4. The implantable medical device lead of claim 1, wherein the at least one cohesive structure is configured to continuously fill a region between adjacent turns of the helically coiled conductor, the region defined by the outer surfaces of the one or more filars bordering on the region.

5. The implantable medical device lead of claim 1, wherein the portions of the insulative coating are interconnected by polymer chains crossing interfaces between the portions of the insulative coating of the at least one cohesive structure.

6. The implantable medical device lead of claim 1, wherein each of the filars of the helically coiled conductor comprises an insulative coating circumferentially covering the outer surface of each of the filars.

7. The implantable medical device lead of claim 6, wherein the minimum width of the at least one cohesive structure, measured in a direction parallel to the center axis of the helically coiled conductor, is less than the sum of the thicknesses of a first section of the insulative coating covering the outer surface of a first filar which borders on the cohesive structure and of a second section of the insulative coating covering the outer surface of a second filar which borders on the cohesive structure, both of said coating sections facing a center axis of the helically coiled conductor.

8. The implantable medical device lead of claim 6, wherein the minimum width of the at least one cohesive structure, measured in a direction parallel to the center axis of the helically coiled conductor, is greater than the sum of the thicknesses of a first section of the insulative coating covering the outer surface of a first filar which borders on the cohesive structure and of a second section of the insulative coating covering the outer surface of a second filar which borders on the cohesive structure, both of said coating sections facing a center axis of the helically coiled conductor.

9. The implantable medical device lead of claim 1, wherein only one of any two adjacent filars comprises an insulative coating circumferentially covering the outer surface of the filar.

10. The medical device lead of claim 9, wherein the minimum width of the at least one cohesive structure, measured in a direction parallel to the center axis of the helically coiled conductor, is less than the thickness of a section of the insulative coating facing a center axis of the helically coiled conductor and covering the outer surface of a filar which borders on the cohesive structure.

11. The medical device lead of claim 9, wherein the minimum width of the at least one cohesive structure, measured in a direction parallel to the center axis of the helically coiled conductor, is greater than the thickness of a section of the insulative coating facing a center axis of the helically coiled conductor and covering the outer surface of a filar which borders on the cohesive structure.

12. The implantable medical device lead of claim 1, wherein the at least one cohesive structure is co-radially and co-axially coiled with the one or more filars.

13. The implantable medical device lead of claim 1, wherein a minimum width of the at least one cohesive structure, measured in a direction parallel to the center axis of the helically coiled conductor, is in the range of about 0.0005 inch to about 0.008 inch.

14. The implantable medical device lead of claim 1, wherein the insulative coating comprises a polymer, a thermoplastic or a thermoplastic elastomer, expanded polytetrafluoroethylene (ePTFE), layered ePTFE, polytetrafluoroethylene (PTFE), polyethylene terephthalate (PETE), ethylene/tetrafluoroethylene copolymer (ETFE), fluorinated ethylene propylene (FEP), polyether ether ketone (PEEK), polyamides, polyimides, para-aramid synthetic fibers, and polyurethane.

15. The implantable medical device lead of claim 1, and further comprising:
a polymer sheath formed about the helically coiled conductor.

16. The implantable medical device lead of claim 15, wherein the polymer sheath comprises a material different than the insulative coating.

17. The lead of claim 16, wherein the material of the polymer sheath has a melting temperature or a glass transition temperature lower than a melting temperature or a glass transition temperature of the insulative coating.

18. The implantable medical device lead of claim 1, and further comprising:
a fixation device at the distal end of the lead body connected to a distal end of the helically coiled conductor.

19. A medical device lead conductor comprising:
at least one helically coiled conducting filar comprising a plurality of filar turns;
at least one of any two adjacent filar turns having a coating, said coating circumferentially covering the outer surface of the at least one filar turn with at least one coating material;
at least one cohesive structure bordering the outer surfaces of any two adjacent filar turns, said at least one cohesive structure consisting of merged portions of the at least one coating material, wherein said at least one cohesive structure is configured to interconnect pairs of adjacent filar turns and to increase the torsional stiffness of the at least one helically coiled conducting filar.

20. A method for producing a helically coiled conductor for a medical device, the method comprising:
forming an insulative coating over at least one of one or more filars;
coiling the one or more filars into a plurality of co-radial turns; and
softening the insulative coating such that adjacent turns of the one or more filars interconnect with one another.

21. The method of claim 20, wherein after coiling and prior to softening the method further comprises:
forming a sleeve around an outer diameter of the one or more coiled filars.

22. The method of claim 21, wherein the sleeve is configured to exert radial compression forces on the one or more filars during the softening step and/or to enhance a flow of portions of the insulative coating into regions located between adjacent turns during the softening step.

23. The method of claim 20, wherein the softening step softens the insulative coating such that portions of the insulative coating flow into regions located between adjacent turns and accumulating in these regions thereby forming at least one coherent structure interconnecting the adjacent turns.

24. The method of claim 20, further comprising the step of forming a polymer sheath over the at least one coiled filar.
